(19) United States
(12) Patent Application Publication
(10) Pub. No.: US 2011/0218622 A1
(43) Pub. Date: Sep. 8, 2011

(54) INDUCTION ACTIVATION OF ADJUSTABLE ANNULOPLASTY RINGS AND OTHER IMPLANTABLE DEVICES

(75) Inventors: Samuel M. Shaolian, Newport Beach, CA (US); Ross Tsukashima, San Diego, CA (US); Brian C. Gray, Lake Forest, CA (US); Donald P. Kannenberg, Irvine, CA (US)

(73) Assignee: MICARDIA CORPORATION, Irvine, CA (US)

(21) Appl. No.: 13/040,657

(22) Filed: Mar. 4, 2011

Related U.S. Application Data
(60) Provisional application No. 61/311,179, filed on Mar. 5, 2010.

(51) Int. Cl. A61F 2/24; A61B 17/00

(52) U.S. Cl. 623/2.37; 128/898

(57) ABSTRACT

Systems and methods to adjust an adjustable medical device that is implanted subcutaneously within the body of a patient. The adjustable medical device is coupled to an adjustment mechanism configured to, when powered, effect a desired adjustment to the adjustable medical device. The adjustment mechanism is electrically coupled to a receiving coil configured to resonate at a desired frequency such that an electric current induced in the receiving coil powers the adjustment mechanism. An induction activation system is configured to utilize magnetic resonance to wirelessly activate the adjustable medical device assembly, from outside the patient's body, through a skin barrier of the patient. The induction activation system comprises a power source and a delivery coil. The power source creates an alternating electrical signal. The delivery coil is electrically coupled to the power source and configured to resonate in response to the alternating electrical signal created by the power source, and thereby generate a resonating magnetic field. The delivery coil is tuned to have a resonant frequency that is the same as a frequency of the alternating electrical signal created by the power source. The receiving coil can also be tuned to resonate at the resonant frequency of the delivery coil. When the delivery coil is positioned near the patient's body, such that the receiving coil is within the magnetic field generated by the delivery coil, an electric current is induced in the receiving coil to drive the adjustment mechanism and thereby effect an adjustment of the adjustable medical device.

### RELATED APPLICATIONS

This application claims the benefit under 35 U.S.C. §119(e) of U.S. Provisional Patent Application No. 61/311,179, filed Mar. 5, 2010, and titled “INDUCTION ACTIVATION OF ADJUSTABLE ANNULOPLASTY RINGS AND OTHER IMPLANTABLE DEVICES,” which is hereby incorporated herein by reference in its entirety.

### BACKGROUND

Disclosed herein are systems and methods directed to adjusting dynamically adjustable annuloplasty rings and other adjustable medical devices that are implanted within a patient.

Heart valve defects, such as regurgitation, may be caused by a relaxation of the tissue surrounding the valve. This causes the valve opening to enlarge, which prevents the valve from sealing properly. Such heart conditions are commonly treated by a procedure during which an annuloplasty ring is sewn around the valve. Synching the tissue to the ring restores the valve opening to its approximate original size and operating efficiency. The proper degree of synching, however, is difficult to determine during open heart surgery. This is due to the fact that the patient is under general anesthesia, in a prone position, with the chest wide open, and that there is a large incision in the heart. These factors affect the normal position and shape of the structures of the heart, including the shape and position of the valve that is repaired during the procedure. Thus, once the incision in the heart is sewn back together, the chest is closed, and other factors affecting the position and shape of the valve are removed, the shape and/or positioning of the annuloplasty ring and/or the synching of the tissue may not be appropriate to provide a desired repair of the valve. Even if the sewing of the annuloplasty ring and synching of the tissue around the annuloplasty ring is done well, the tissue may continue to relax over the patient's lifetime, such that the heart condition returns. Therefore, adjusting the shape and/or position of the implanted annuloplasty ring, post-procedure, may be desirable.

### SUMMARY

Disclosed herein are systems and methods directed to adjusting a dynamically adjustable annuloplasty ring or other adjustable medical device that is implanted within a patient, post-procedure and over the patient's lifetime.

### BRIEF DESCRIPTION OF THE DRAWINGS

Understanding that drawings depict only certain embodiments and are not therefore to be considered to be limiting in nature, non-limiting and non-exhaustive embodiments of the disclosure are described and explained with additional specificity and detail through the use of the accompanying drawings, in which:

FIG. 1A is a circuit diagram of an implantable dynamically adjustable annuloplasty ring assembly, according to one embodiment.

FIG. 1B illustrates circuitry of an external (to the patient) radio frequency powered (RF) induction activation system, according to one embodiment.

FIG. 2 is a block diagram of a system for inductively activating a dynamically adjustable annuloplasty ring, according to one embodiment.

FIG. 3 is a block diagram of additional circuitry implanted in a patient with a dynamically adjustable annuloplasty ring, according to one embodiment.

FIG. 4 is a block diagram of a display panel or user interface for use with an RF induction activation system, according to one embodiment.

FIG. 5 illustrates an adjustable annuloplasty ring and heating element, according to one embodiment.

FIGS. 6A and 6B illustrate embodiments of annuloplasty rings and heating elements, according to various embodiments.

FIG. 7 illustrates an adjustable annuloplasty ring and heating element, according to another embodiment.

FIG. 8 illustrates an adjustable annuloplasty ring and heating element, according to still another embodiment.

FIG. 9 illustrates a variable pitch power heating element, according to one embodiment.

### DETAILED DESCRIPTION OF EMBODIMENTS

Disclosed herein are systems and methods directed to utilizing magnetic resonance to provide power to dynamically adjustable annuloplasty rings or other adjustable medical devices that are implanted within a patient. Using the disclosed systems and methods, power levels of 10 Watts or more, for example, can be easily transmitted to thereby power implanted medical devices without the need for a direct physical electrical connection.

A dynamically adjustable annuloplasty ring allows for adjustment to achieve a proper shape and/or proper degree of synching both during open heart surgery and over the patient's lifetime, to treat valve regurgitation. A dynamically adjustable annuloplasty ring may include, for example, shape memory material such as Nitinol. Such annuloplasty rings may be reshaped by heating the shape memory material according to certain embodiments disclosed herein. Adjustment of the adjustable annuloplasty ring can be done at early onset of recurring regurgitation, with no discomfort to the patient, to stop disease progression with just a simple procedure without hospital stay requirement, and without a need for an invasive procedure or prolonged anesthesia. The systems and methods disclosed herein may be used to treat mitral regurgitation and tricuspid regurgitation using similar construction, design, and numbers of components.

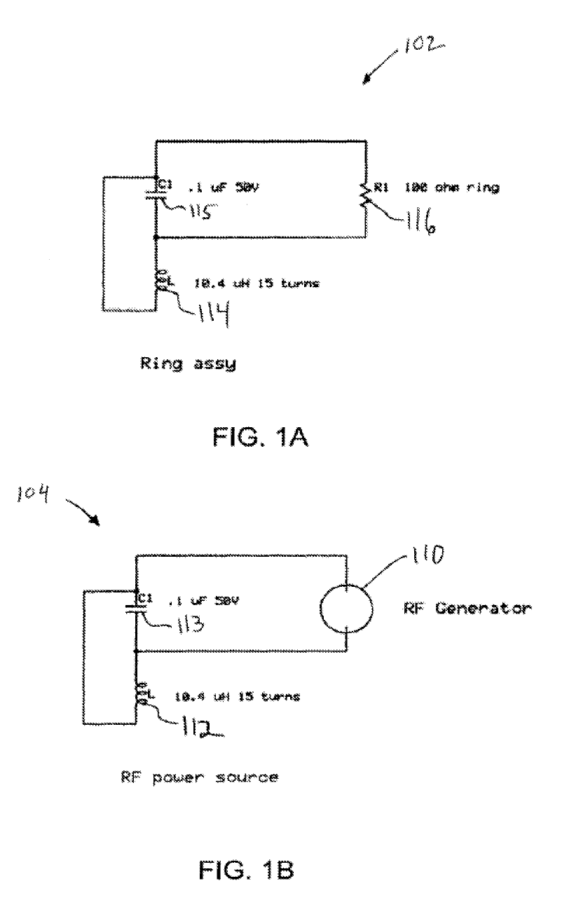

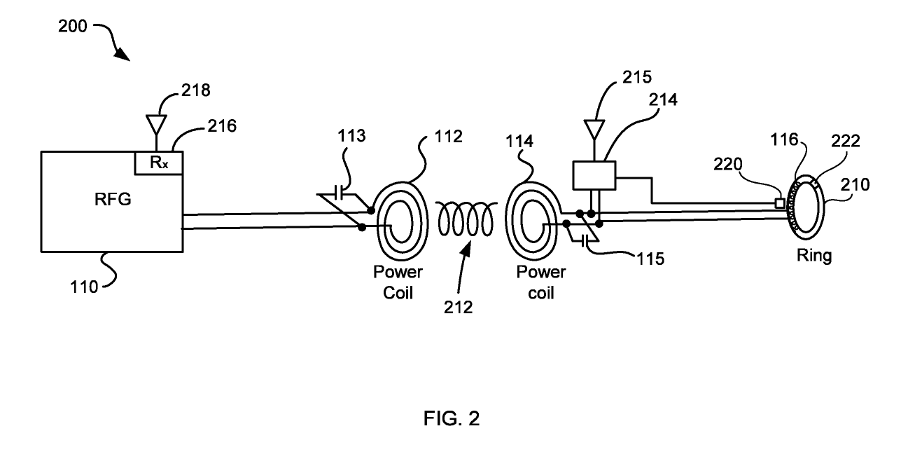

FIG. 1A illustrates circuitry of an implantable dynamically adjustable annuloplasty ring assembly 102 and FIG. 1B illustrates circuitry of an external (i.e., external to the patient) radio frequency powered (RF) induction activation system 104, according to one embodiment. FIG. 2 is a block diagram of a system 200 for inductively activating an annuloplasty ring 210 according to certain embodiments.

Referring to FIGS. 1A, 1B, and 2, the radio frequency powered (RF) induction activation system 104 includes a power source 110 (also referred to herein as an RF generator or RFG) capable of creating an alternating electrical signal of suitable power. The power source 110 is connected to a delivery coil 112 tuned to resonate at the same frequency as the output of the power source 110. A capacitor 113 is used to tune the delivery coil 112 to resonate at the desired frequency. The implantable dynamically adjustable annuloplasty ring assembly 102 comprises a second (receiving) coil 114, positioned within the patient, that is designed to resonate at substantially the same frequency as that of the delivery coil 112 connected to the power source 110. A capacitor 115 is used to tune the receiving coil 114 to resonate at the desired frequency. The receiving coil 114 is connected to a heating element 116 (represented by a resistance R1 in FIG. 1A) in the annuloplasty ring 210 (shown in FIG. 2). To activate the annuloplasty ring 210, the delivery coil 112 is placed near the receiving coil 114 of the annuloplasty ring 210 (e.g., near the patient's chest) and switched on. Power from the resonating magnetic field 212 (shown in FIG. 2) is then inductively transferred across the skin barrier to the receiving coil 114 and converted to electrical current that is subsequently used to heat the annuloplasty ring 210. In an example embodiment, the inductance frequency is above about 100 kHz so that any leakage current that may come in contact with the patient, would not cause uncomfortable sensations during activation.

In certain embodiments, embedded computing and/or remote temperature sensing is used. For example, FIG. 2 shows that additional circuitry 214 may be implanted in the patient. As discussed below, the additional circuitry 214 may include transmitter circuitry (including an antenna 215), a microprocessor, power circuitry, and temperature measuring circuitry (e.g., one or more thermocouple (TC) devices 220, coupled to the additional circuitry 214). Similarly, the RFG 110 may include receiver circuitry 216 (including an antenna 218) for receiving temperature and other data from the additional circuitry 214 implanted in the patient. Although not shown, the RFG 110 may also include a processor for processing and displaying the information received from the additional circuitry 214 implanted within the patient.

The information received from the additional circuitry 214 may include, for example, the power induced in the annuloplasty ring 210. In one embodiment, the power transferred to the annuloplasty ring 210 is measured by reading the voltage across the annuloplasty ring 210 and/or heating element 116 and, because the resistance of the annuloplasty ring 210 and/or heating element 116 is known, the power can be calculated and communicated to the RFG 110 by the telemetry link. In another example, the temperature and size of the annuloplasty ring 210 may be sensed and sent by transmitter circuitry in the additional circuitry 214 to the receiving circuitry 216 via radiotelemetry. Temperature may be sensed using a thermocouple device 220, and the size of the ring may be deduced via built in strain gauges 222 (e.g., different resistance values equal a proportional change in size).

In one embodiment, the RFG 110 automatically finds a resonant point. The RFG 110 may be programmed to analyze wattage delivered during operation (e.g., as discussed above) and may adjust the output frequency to increase or maximize the greatest power transfer. This may be accomplished in certain embodiments by directly monitoring the current output on the delivery coil 112, or the peak voltage induced in the receiving coil 114 via telemetry.

In one embodiment, the system 200 is capable of multiple resonant frequencies. For example, the heating element 116 (coupled to the annuloplasty ring 210) may be electrically connected to more than one coil—each coil having a different natural resonance. In another embodiment, different coils may be attached to different heating elements or devices in the annuloplasty ring 210 that can be operated separately. The transmitting power source 110 may have a set of coils (e.g., including the delivery coil 112) that can be selectively used to couple to its respective sister coil (e.g., including the receiving coil 114) coupled to the annuloplasty ring 210.

By using this wireless technique of power transmission, the patient may be electrically isolated from the system 200 during activation of an implanted device. Thus, the possibility of electrocution due to a ground fault is eliminated or reduced.

In some embodiments, centering of coils is used. Such embodiments use techniques of aligning the coils, such as through the use of physical landmarks molded into a housing of the implanted receiving coil, magnets, and/or infrared lighting. For example, an infrared light emitting diode (LED) may be installed on the implanted receiving coil 114 and may light during activation. An infrared detector located on the delivery coil 112 may be configured to give a user feedback on how much light it receives. A set of magnets may also be strategically placed in the delivery coil 112 and receiving coil 114. As the magnets are brought close together, the magnetic attraction may be utilized to align the coils 112, 114.

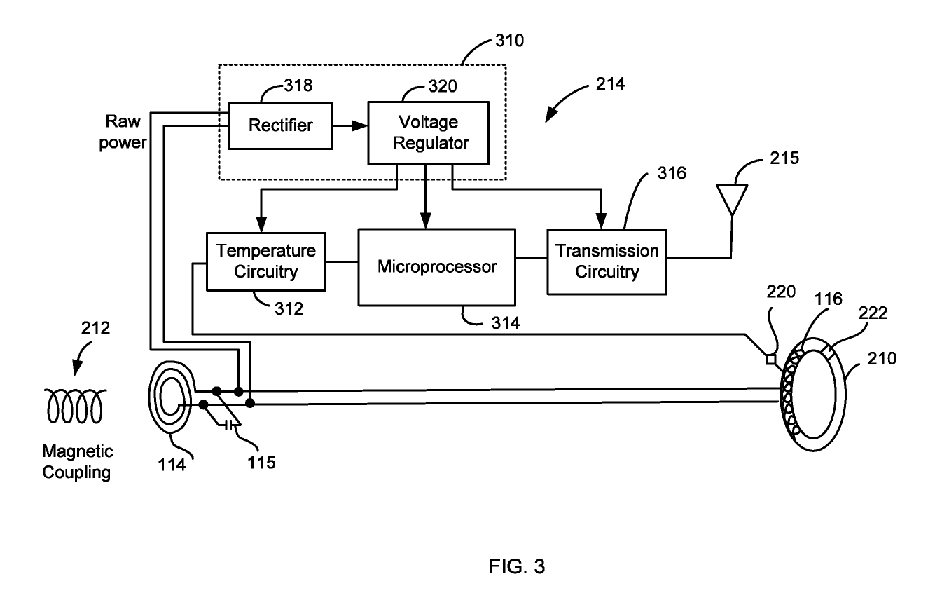

FIG. 3 is a block diagram illustrating the additional circuitry 214 of the implantable dynamically adjustable annuloplasty ring assembly 102, according to one embodiment. The additional circuitry 214 includes power circuitry 310, temperature circuitry 312 comprising a TC analog circuit, a microprocessor 314 (or CPU), and transmission circuitry 316. The power circuitry 310 includes a rectifier 318 and a voltage regulator 320. By rectification, DC power can be created to power the electrical circuit of the ring assembly 102. The DC power can power the embedded CPU 314. The DC power can also power the temperature circuitry 312, to monitor temperature of the annuloplasty ring 210 during activation. The DC power can also power the transmission circuitry, to transmit temperature information (and any other information) wirelessly to the power source 110, thereby providing a closed loop feedback system. A “thermostat” type circuit may also be used to automatically break the heating circuit if the temperature exceeds a certain threshold value. In addition to temperature, other information transmitted from the additional circuitry 214 may include, for example, a unique code or value that identifies the particular annuloplasty ring 210, a current size of the annuloplasty ring 210, and/or change in ring size. The initial ring size may be known and may be programmed into the device. For example, the initial ring size may be programmed into a non-volatile memory of the CPU 314 and sent to the induction activation system 104 via the telemetered data from the transmission circuitry 316 to the receiver circuitry 216 (FIG. 2).

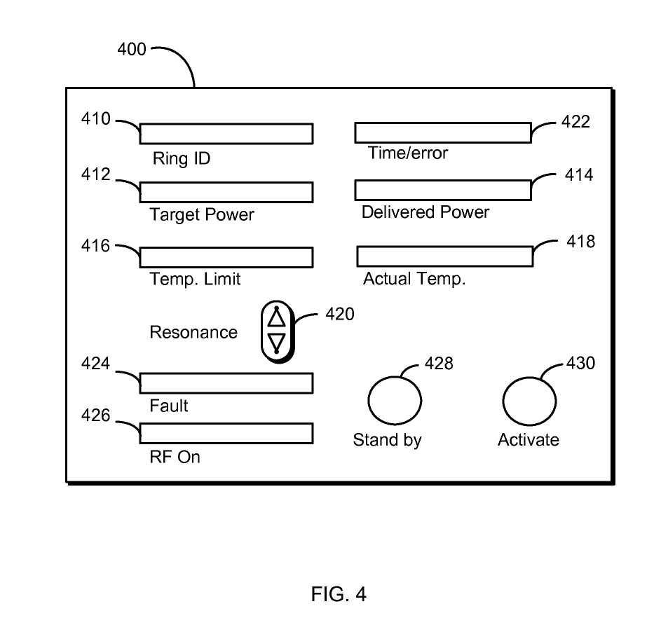

FIG. 4 is a block diagram of a display panel 400 or user interface for use with the RFG 110, according to one embodiment. The display panel 400 includes a display field for a ring identifier (ID) 410, a display field for target power 412, a display field for determined or measured power 414, a display field for temperature limit 416, a display field for actual or measured temperature 418, a user control for setting the resonant frequency 420, a display field for time and/or error indicators 422, a fault indicator 424, an RF On indicator 426, a standby indicator 428, and an activate user control 430.

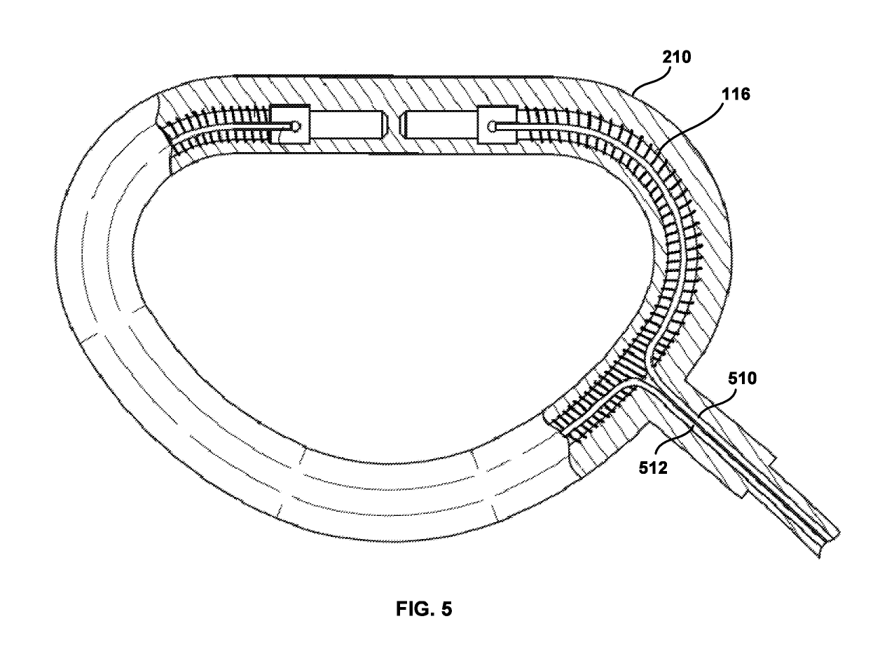

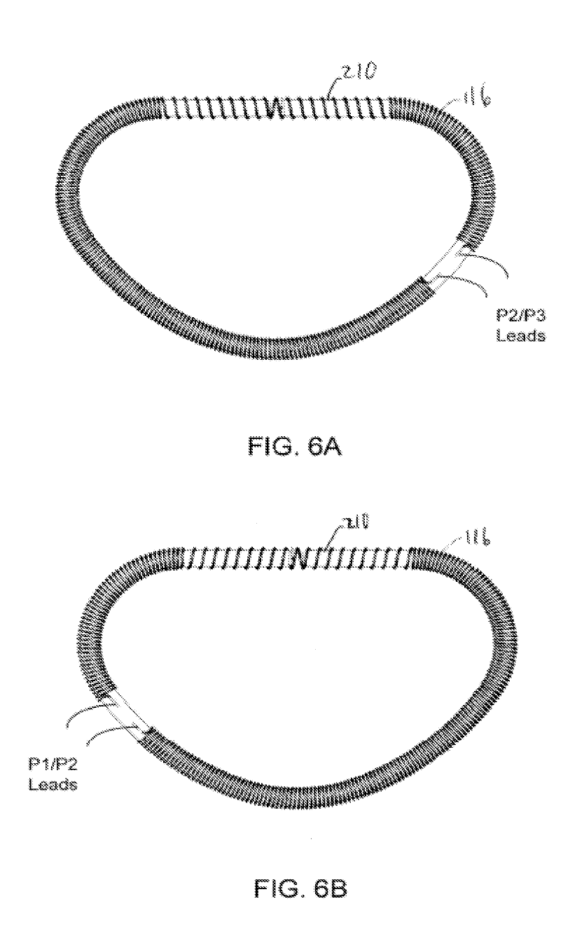

FIG. 5 illustrates an adjustable annuloplasty ring 210 and heating element 116 according to one embodiment. Leads 510, 512 for providing induced current through the heating element 116 are also shown. FIGS. 6A and 6B show embodiments of the annuloplasty ring 210 and heating elements according to various embodiments that allow the leads to exit through the septal wall, the right atrium subclavian vein, or both leads may follow the ring contour and exit at P₁/P₂ leaflet junction or P₃/P₂ leaflet junction.

In certain embodiments, the receiving coil 114 (shown in FIGS. 2 and 3) and any associated internal circuitry may be placed anywhere within the patient and outside the heart of the patient. For example, the receiving coil 114 and/or additional circuitry 214 may be implanted immediately below the surface of the skin and couple to the heating element 116 (coupled to the annuloplasty ring 210) via one or more wires extending into the heart. In another embodiment, the receiving coil 114 and associated internal circuitry may be integrated with the annuloplasty ring. For example, the receiving coil 114 and additional circuitry 214 may be incorporated internal to the annuloplasty ring 210. In still another embodiment, the receiving coil 114 may be implanted adjacent the lead wire and/or the receiving coil, in close proximity to the annuloplasty ring.

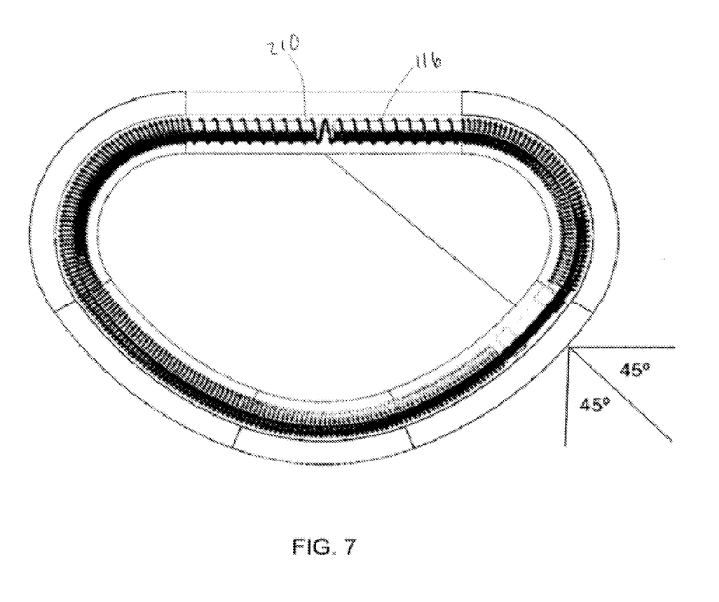

FIG. 7 illustrates an adjustable annuloplasty ring 210 and heating element 116, according to another embodiment. The design illustrated in FIG. 7 allows angles of an exiting lead to be altered from 0° to 90°. This allows a surgeon flexibility, when implanting the annuloplasty ring 210, to select an exit location that avoids major arteries near the myocardial wall near the P₃/P₂ leaflet junction. Further, the exiting lead wire can be designed so that a surgeon can select an “above the Nitinol core wire” model or a “below the Nitinol core wire” model that fits the annulus sizing criteria for best placement of the subcutaneous lead wire.

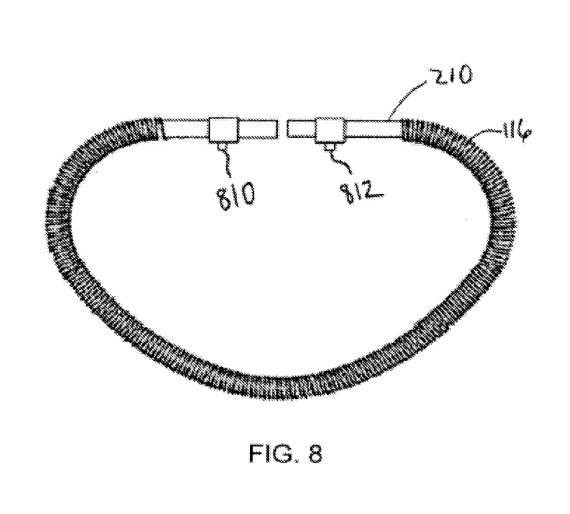

FIG. 8 illustrates adjustable annuloplasty ring 210 and heating element 116, according to still another embodiment. FIG. 8 illustrates a heating element 116 design-variation. The illustrated embodiment of a heating element 116 provides full heating energy in the P₃/P₂ leaflet area. Also, lead wires (not shown) may be routed from contact points 810, 812 to exit at the P₃/P₂ leaflet area, according to certain embodiments. Such embodiments may use thin wall insulating tubes on the lead wires to reduce the crossing profile.

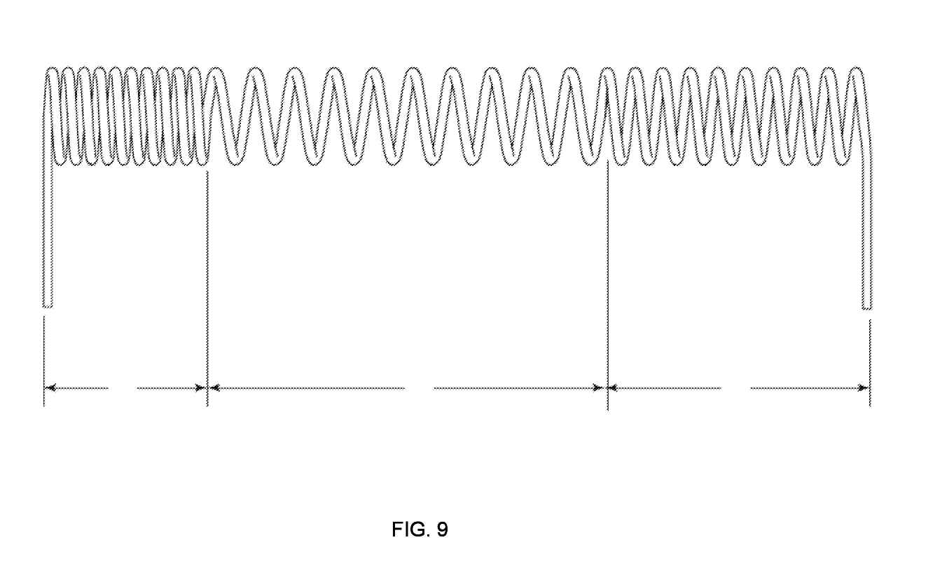

FIG. 9 illustrates a variable pitch power heating element, according to one embodiment. For the illustrated sections A, B and C, the number of turns in each section, the overall wire length in each section, and the impedance of each section may be selected based on application to a particular annuloplasty ring. In one embodiment, the wire is wound over a 0.057 inch +/−0.001 inch mandrel or pin gauge. In one embodiment, the coil sections A, B, and C have consistent pitches and do not overlap.

As can be appreciated, in other embodiments, the heating element 116 may be substituted for an alternative adjustment mechanism, such as a motor. The dynamically adjustable annuloplasty ring assembly 102 may comprise an annuloplasty ring 210 having a motor to drive adjustment of the size and/or shape of the ring. The receiving coil 114 may be electrically coupled to the motor such that a current induced in the receiving coil 114 may power the motor. The additional circuitry 214 of the ring assembly 102 may detect and transmit information about the shape of the annuloplasty ring 210 and operation of the motor.

In still other embodiments, the adjustable medical device may be a device other than an annuloplasty ring. For example, the adjustable medical device may comprise an artificial pacemaker. The pacemaker may include a battery that is electrically coupled to charging circuitry and to the receiving coil 114. The power induced in the receiving coil 114 can be used to charge the battery of the pacemaker. The pacemaker may also be programmable to adjust the frequency of the electric impulses delivered to the heart muscles to regulate the beating of the heart. The power induced in the receiving coil 114 may be used to reprogram the pace of the pacemaker.

It will be understood by those having skill in the art that many changes may be made to the details of the above-described embodiments without departing from the underlying principles of the invention. The scope of the present invention should, therefore, be determined only by the following claims.

1. A system to adjust an adjustable medical device, the system comprising:
an adjustable medical device assembly that is implantable subcutaneously within a body of a patient, the adjustable medical device assembly comprising:
an adjustable medical device;
an adjustment mechanism coupled to the adjustable medical device and configured to, when powered, effect an adjustment to the adjustable medical device; and
a receiving coil electrically coupled to the adjustment mechanism and configured to resonate at a desired frequency such that an electric current induced in the receiving coil provides power to the adjustment mechanism; and
an induction activation system configured to utilize magnetic resonance to wirelessly activate the adjustable medical device assembly, from outside the patient's body, through a skin barrier of the patient, the induction activation system comprising:
a power source configured to create an alternating electrical signal of suitable power to resonate a coil; and
a delivery coil electrically coupled to the power source and configured to resonate in response to the alternating electrical signal created by the power source and thereby generate a resonating magnetic field, wherein the delivery coil is tuned to have a resonant frequency that is the same as a frequency of the alternating electrical signal created by the power source,
wherein the receiving coil of the adjustable medical device assembly is tuned to resonate at the resonant frequency of the delivery coil, and wherein positioning the delivery coil outside of the body of the patient and within proximity to the receiving coil positioned internal to the body of the patient, such that the receiving coil is within the magnetic field generated by the delivery coil, induces an electric current in the receiving coil that drives the adjustment mechanism to effect an adjustment of the adjustable medical device.

2. The system of claim 1, wherein the adjustable medical device is a dynamically adjustable annuloplasty ring including a shape memory material configured to reshape in response to heating, and wherein the adjustment mechanism comprises a heating element configured to heat the annuloplasty ring to effect an adjustment thereof.

3. The system of claim 2, the adjustable medical device assembly further comprising a strain gauge and circuitry configured to monitor changes in the size of the dynamically adjustable annuloplasty ring as it reshapes.

4. The system of claim 1, the adjustable medical device assembly further comprising temperature circuitry configured to monitor a temperature of the adjustable medical device, wherein the adjustable medical device is configured to adjust in response to heat.

5. The system of claim 1, the adjustable medical device assembly further comprising transmission circuitry configured to transmit information about the adjustable medical device to the induction activation system, the induction activation system further comprising receiving circuitry configured to receive information transmitted from the transmission circuitry.

6. The system of claim 1, the adjustable medical device assembly further comprising a landmark configured to aid in centering the delivery coil with the receiving coil, the induction activation system further comprising a detector to detect the landmark to aid in centering the delivery coil with the receiving coil.

7. The system of claim 6, wherein the landmark is an infrared light source and the detector comprises an infrared detector configured to provide feedback on the amount of infrared light received from the infrared light source during alignment of the delivery coil with the receiving coil.

8. The system of claim 6, wherein the landmark comprises a first magnet and the detector comprises a second magnet, and wherein magnetic attraction between the first magnet and the second magnet aligns the delivery coil with the receiving coil.

9. A method for dynamically adjusting an adjustable medical device implanted in a body of a patient, the method comprising:
implanting an adjustable medical device assembly subcutaneously within a body of a patient, the adjustable medical device assembly comprising:
an adjustable medical device;
an adjustment mechanism coupled to the adjustable medical device and configured to, when powered, effect an adjustment to the adjustable medical device; and
a receiving coil electrically coupled to the adjustment mechanism and configured to resonate at a desired frequency such that an electric current induced in the receiving coil provides power to the adjustment mechanism; and
inducing an electric current in the receiving coil to power the adjustment mechanism and effect a desired adjustment of the adjustable medical device.

10. The method of claim 9, wherein inducing the electric current is accomplished post-operatively, after the adjustable medical device has been implanted, and through the skin barrier of the patient.

11. The method of claim 9, wherein inducing an electric current in the receiving coil comprises:
positioning an induction activation system to induce the electric current, the induction activation system comprising:
a power source configured to create an alternating electrical signal of suitable power to resonate a coil; and
a delivery coil electrically coupled to the power source and configured to resonate in response to the alternating electrical signal created by the power source and thereby generate a resonating magnetic field, wherein the delivery coil is tuned to resonate at a resonant frequency that is the same frequency as the alternating electrical signal created by the power source, and wherein the receiving coil of the adjustable medical device assembly is tuned to resonate at the resonant frequency of the delivery coil,
wherein the induction activation system is positioned on the outside of the body of the patient such that the receiving coil within the body of the patient is within the resonating magnetic field generated by the delivery coil, and the resonating magnetic field induces an electric current in the receiving coil that drives the adjustment mechanism to adjust the adjustable medical device.

12. The method of claim 11, wherein the induction activation system is configured to utilize magnetic resonance to wirelessly activate the adjustable medical device assembly through the skin barrier.

13. The method of claim 11, wherein the adjustable medical device further comprises a landmark configured to aid in centering the delivery coil with the receiving coil, the induction activation system further comprises a detector to detect the landmark to aid in centering the delivery coil with the receiving coil, and wherein the method further comprises aligning the delivery coil and receiving coil by detecting the landmark with the detector.

14. The method of claim 9, the adjustable medical device further comprising temperature circuitry configured to monitor a temperature of the adjustable medical device, the method further comprising:
modifying electric current induced in the receiving coil based on temperature information gathered by the temperature circuitry.

15. The method of claim 9, the adjustable medical device further comprising sizing circuitry configured to monitor a size and shape of the adjustable medical device, the method further comprising:
modifying electric current induced in the receiving coil based on size or shape information gathered by the sizing circuitry.

16. The method of claim 9, the adjustable medical device further comprising transmission circuitry configured to transmit information about the adjustable medical device, the method further comprising:
receiving from the transmission circuitry temperature information about the adjustable medical device.

17. The method of claim 16, further comprising receiving from the transmission circuitry one of size and shape information about the adjustable medical device.

18. The method of claim 9, wherein the adjustable medical device is a dynamically adjustable annuloplasty ring including a shape memory material configured to reshape in response to heating, and wherein the adjustment mechanism comprises a heating element configured to heat the annuloplasty ring to effect an adjustment thereof.
